(19) United States
(12) Patent Application Publication
(10) Pub. No.: US 2022/0254506 A1
(43) Pub. Date: Aug. 11, 2022

(54) EXTENDED REALITY SYSTEMS AND METHODS FOR SPECIAL NEEDS EDUCATION AND THERAPY

(71) Applicants: Joseph Anthony Pillitteri, Colorado Springs, CO (US); Ginger Diane Pillitteri, Colorado Springs, CO (US)

(72) Inventors: Joseph Anthony Pillitteri, Colorado Springs, CO (US); Ginger Diane Pillitteri, Colorado Springs, CO (US)

(21) Appl. No.: 17/494,656

(22) Filed: Oct. 5, 2021

Related U.S. Application Data
(63) Continuation-in-part of application No. 17/163,075, filed on Jan. 29, 2021.
(60) Provisional application No. 62/968,868, filed on Jan. 31, 2020.

(51) Int. Cl. G16H 50/70; G16H 10/60; G06T 19/00

(52) U.S. Cl. CPC G16H 50/70; G06T 19/006; G16H 10/60

(57) ABSTRACT

Some implementations can include a computer-implemented method, computer readable media and/or system for remotely providing and/or monitoring special needs educational and/or therapeutic services.

### RELATED APPLICATIONS

This application is a continuation in part of U.S. application Ser. No. 17/163,075, Entitled “Systems, Methods And Computer Readable Media For Special Needs Service Provider Matching,” filed on Jan. 29, 2020, which claims priority to U.S. Provisional Application No. 62/968,868, entitled “Systems, Method and Computer Readable Media for Special Needs Service Provider Matching,” and filed on Jan. 31, 2020, both of which are incorporated herein by reference in their entirety.

### FIELD

Some implementations relate to the field of extended reality computer systems. More specifically, some implementations relate to extended reality systems and methods to provide and/or monitor special needs educational or therapeutic services.

### BACKGROUND

Families having a child or other family member whose has a special need based on a condition or medical diagnosis often have a need for service providers that are experienced and able to provide a service to those with one or more special need conditions or diagnoses. A person with special needs can include a child or an adult who has been detected with some form of physical, cognitive or learning disabilities such as Autism spectrum disorder (ASD), dyslexia, processing disorder, physical, hearing, or visual impairment, and may require a learning environment that is specifically designed for that person. It may be difficult or impossible to determine via computerized search whether a service provider has such experience and accepts clients with one or more conditions or diagnoses.

Further, there may not exist a rating or review system for service providers that provide a rating for the service provider on both the core service provided by the service provider and the service provider's ability to work with a person with special needs. The rating can include a star rating (or other symbolic rating) and/or a percentage rating.

Also, there may exist a need for locating service providers to assist a person with special needs or the family of a person with special needs in a town or location familiar or unfamiliar to the family of the person with special needs. Families of the person with special needs may be unable to locate a service provider that 1) can accommodate the special needs of the family member, and 2) has been reviewed by others and received a favorable rating.

Further, in some scenarios, a service provider may need to work remotely with a person with special needs. Online platforms (e.g., video conferencing services such as Zoom) may not work well for individuals with special needs. Some students and people have significant special needs and may not understand what they are being shown or taught in typical virtual formats such as video or audio conferencing.

In some instances, children with special needs may have problems coping with the traditional learning environment. For example, they may find it difficult to follow instructions, lessons, even their textbooks. Further, they may need personal attention from the teacher, and may be unable to handle the pressure of competition.

Also, these children can be subjected to bullying from other students, which can further discourage them. A need may exist for special tools and lessons that are designed for them.

Some implementations were conceived in light of the above-mentioned problems and limitations, among other things.

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

Some implementations can include a method comprising obtaining user registration data, obtaining service provider registration data, and receiving a query. The method can also include matching a combination of query data and user registration data with the service provider data, and outputting a result of the matching, wherein the result includes one or more records that match within a given threshold.

The method can further include filtering the result of the matching based on one or more third-party data items and outputting the filtered results. In some implementations, the user registration data includes one or more of a diagnosis or a condition corresponding to a special need of a person. In some implementations, the combination includes the diagnosis or the condition. In some implementations, the diagnosis or the condition is independent of a service provided by the service provider, and wherein the matching results include service provider data indicating that the matching service providers provide the service from the query to people having the diagnosis or the condition. In some implementations, the third-party data includes one of a rating above a rating threshold or a verification, wherein the rating threshold is included as part of the query, and the verification indicates that the service provider was verified by a third-party.

### BRIEF DESCRIPTION OF THE DRAWINGS

FIG. 1 is a block diagram of an example special need service provider matching system in accordance with some implementations.

FIG. 2 is a flowchart for special needs service provider matching in accordance with some implementations.

FIG. 3 is a block diagram of an example computing device in accordance with some implementations.

FIG. 4 is a block diagram of an example system for remotely providing and monitoring services for individuals with special needs via extended reality systems in accordance with some implementations.

FIG. 5 is a block diagram of an example system for remotely providing and monitoring extended reality services for individuals with special needs in accordance with some implementations.

FIG. 6 is a flowchart for providing extended reality services for individuals with special needs in accordance with some implementations.

### DETAILED DESCRIPTION

The systems and methods provided herein may overcome one or more deficiencies of some conventional methods of providing education or therapy services to persons with special needs.

Some implementations can include a method or system to search for services for a person with special needs using a combination of one or more diagnoses, age, location (and possibly other demographic data), where the service is independent of a diagnosis (e.g., finding a barber for a person with autism). Some implementations can include searching using a current location or a selected location (e.g., for vacation, relocation research, etc.). Some implementations can include presenting a semi-personalized view of a web site (or section of website, e.g., a “Selected for You” type section) for each visitor, where view is based on location, one or more diagnoses, age, history of use, etc. Some implementations can include alerts to members about new reviews related to diagnoses that the user has indicated in a profile, e.g., alerts that are tailored to specific combination of data such as a diagnosis/age/location combination.

Some implementations can include a verification of service providers, e.g., credential review, checking with licensing agency, etc., so that a verified provider has a special indication on the site or app.

As used herein diagnosis or condition refers to anything that creates a special need for an individual or caregiver. For example, a diagnosis or condition can include, but is not limited to mental, cognitive, emotional, physical, psychological, medical, neurological, or developmental disabilities or limitations. Some examples of diagnoses and conditions include: autism, deaf-blindness, hearing impairment, visual impairment, deafness, developmental delay, emotional disturbance, intellectual disability, multiple disabilities, orthopedic impairment, other health impairment, specific learning disability, speech or language impairment, traumatic brain injury, visual impairment (including blindness), auditory processing disorder, academic problems, disorders of attention, poor motor abilities, psychological process deficits and information-processing problems, lack of cognitive strategies needed for efficient learning, oral language difficulties, reading difficulties, written language problems, mathematical disorders, social skill deficits and/or deficiencies, sensory sensitivities, dyslexia or other processing disorders, ADD, ADHD, and/or gifted/talented.

In general, a diagnosis or condition can represent any person with a disability or any person evaluated in accordance with any state or federal law as having an intellectual disability, a hearing impairment (including deafness), a speech or language impairment, a visual impairment (including blindness), a serious emotional disturbance (e.g., “emotional disturbance”), an orthopedic impairment, autism, traumatic brain injury, any other health impairment, a specific learning disability, deaf-blindness, or multiple disabilities, and who, by reason thereof, needs special education and related services.

Further, a person with special needs can include anyone who fits any definition found under IDEA (individuals with disabilities education act, Federal) or any government (e.g., federal, state, or local) definition or medical or educational definition of an individual with special needs.

Services provided that can be matched with an implementation of the system described herein can include but are not limited to any service that may require special training, experience, skills, knowledge, abilities or augmentations to deal with, service, handle, or accommodate a person with any type of disability listed above. In some implementations, a doctor's office or other professional can provide a recommendation to the family for any special needs services based upon certain criteria or combinations of criteria, for example diagnosis, characteristics and/or demographic to name a few.

Services can include:
1. Professional services
   a. Optometrist
   b. Audiologist
   c. Dentists
   d. Physician—generalists, specialists, etc.
   e. Psychologist
   f. Lawyers
      i. Trusts
      ii. Accommodations, etc.
   g. ABA
   h. Nutritionist/dietician
      i. Therapist
      i. Speech
      ii. OT
      iii. APE
      iv. PT
      v. Vision therapy
   j. Financial advisors
      i. Financial planners
         1. Trusts
         2. Planning
2. Lifestyle services
   a. Travel
   b. Vacation
   c. Diet/nutrition
   d. Haircuts and other aesthetic services
   e. Activities
3. Transition
   a. Schools
      i. Pre-school
      ii. Elementary School
      iii. Middle School
      iv. High School
      v. College
   b. Services
   c. Employment
   d. Residential
4. Education
   a. Schools
      i. Private
      ii. Public
      iii. College
   b. Tutors
5. Financial
   a. Trusts
   b. Financial planners

Some implementations can include a website, a mobile application, and/or a desktop application. Some implementations can be configured for use with voice activated devices (Alexa, Siri, Google Home, or other smart speaker, etc.), a virtual assistant or any other voice queries and a natural-language user interface to answer questions, make recommendations, and perform actions by delegating requests to a set of Internet services in order to use the information to make recommendations for a user. Artificial intelligence (AI) compatible to learn the functionality, ability, limitations, etc. of a user and make recommendations on maps or other means for services, parks, tools, toys, nutrition, diet, or other recommendations suitable to the person with a special need. For example, if the system has permission to monitor conversations, the system could monitor parents talking about a diagnosis or condition of themselves of a family member. The system can use what info was gathered and use the rating system to automatically recommend a professional or a course of action, a forum, blog, or a discussion group on the website, etc, that has been matched according to the technique described herein using a machine learning model.

In some implementations, resources can be organized into categories (e.g., in a menu structure or other navigation system within an app, website or other software program). The categories can include, but are not limited to, professional, therapist/therapies, lifestyle, support groups, transition, education, camps, programs and activities, evaluation and diagnosis, safety, and state and local organizations.

Within the professional category, service types can include adoption, audiologist, dentists, advocacy (e.g., legal or financial), medical, nutrition, and/or psychologist. Within the therapy/therapies category service types can include applied behavior analysis, early start Denver model, floor time or DIR, local early intervention providers, Medicaid waiver, neurologists, occupational therapy, physical therapy, pivotal response treatment (PRT), relationship development intervention (RDI), speech and language therapy, state developmental disability agency, state early intervention office, TEACCH, verbal behavior, and/or vision therapy.

Within the lifestyle category service types can include activities, diet/nutrition, haircuts, travel, and/or vacation. Within the support groups category, service types can include adults, autism communities, family/parent, grandparents, and/or sibling. Within the transition category service types include employment/post-school, residential, and/or transportation. Within the education category, service types can include schools and/or tutors.

Within the camps, programs, and activities category after-school programs, arts and crafts, camps, community activities, day programs, equine programs, respite care, and/or social skills. Within the evaluation and diagnosis category, autism evaluation, pediatricians/developmental, psychiatrists, psychologists and counselors, and/or specialized autism centers. Within the safety category, service types can lude first responder resources. Within the state and local category, service types can include assistive technologies, local disability organizations, protection and advocacy, special education office, social security office locator, and/or state services and entitlements.

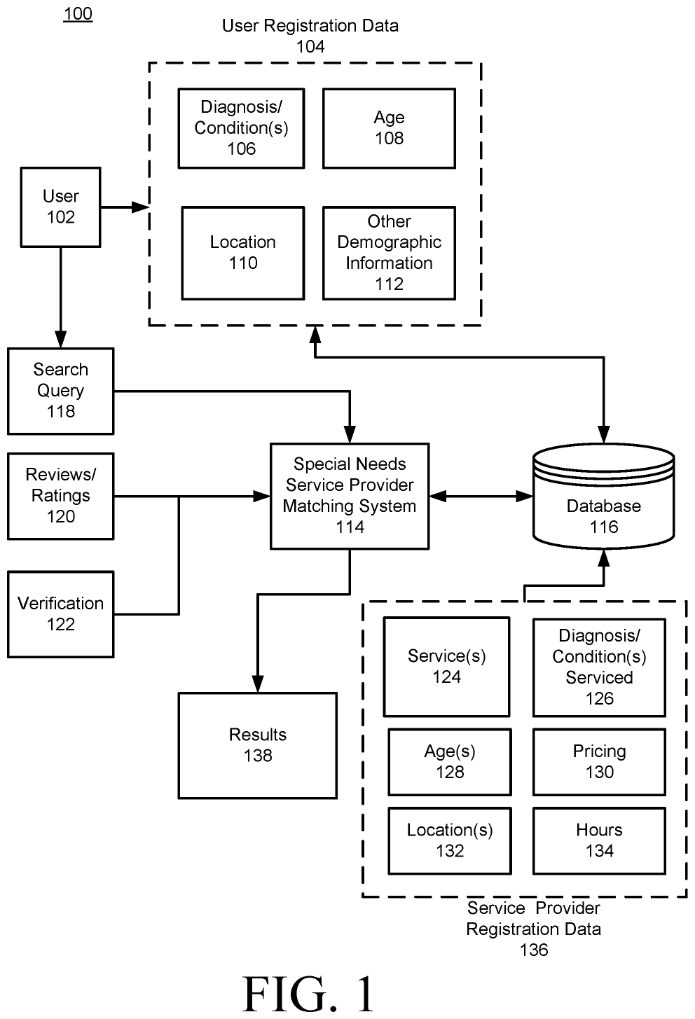

FIG. 1 is a block diagram of an example special need service provider matching system environment 100 in accordance with some implementations. The system environment 100 can include one or more users 102, where each user provides user registration data 104 that can include (for each person with special needs the user registers) one or more diagnoses or conditions 106, an age 108, a location 110, and/or other information 112 (e.g., gender, other medical conditions, abilities, limitations, etc.). The user registration data 104 can be stored in a data store or database 116 via a special needs service provider matching system 114.

The special needs service provider matching system 114 can access the database 116 to store service provider registration data 136, which can include services provided 124, diagnoses or conditions serviced 126, ages 128, pricing 130, location(s) 132, or hours 134.

The special needs service provider matching system 114 also receives a search query 118 from the user 102. The search query can include a service that the user is searching for (e.g., haircut or music lesson). The special needs service provider matching system 114 also receives reviews/ratings 120 of service providers and verifications 122 of service providers.

The special needs service provider matching system 114 matches service providers with user queries based on the method described below to generate results 138. In some implementations, the results can include a match value such as a percentage match and/or a points or star system value that indicates how closely a service provider matches the search query for a given person with special needs in a given area. The matching service providers can be sorted by the match value for display to a user.

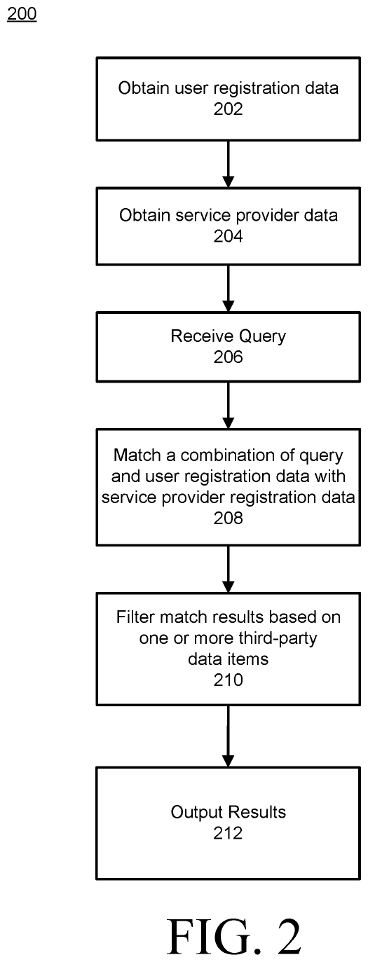

FIG. 2 is a flowchart for special needs service provider matching in accordance with some implementations. Processing begins at 202, where user registration data is received. Processing continues to 204.

At 204, service provider registration data is received. Processing continues to 206.

At 206, a query is received. Processing continues to 208.

At 208, a combination of query and user registration data is matched against service provider registration data. For example, if a user query is haircut and the user registration data for one or more people associated with user registration includes a diagnosis of autism, the system will match against a service provider (barber or hairdresser) that has indicated an ability to work with persons having a diagnosis of autism. The match may be exact or may be within a threshold or a nearest match (e.g., a diagnosis may not be exact but may to a service provider that works with people having a diagnosis or condition similar to that of the user registration data). Processing continues to 210.

At 210, results from 208 are optionally filtered based on one or more third-party data items such as rating or verification. For example, a person may want a service provider with a rating above a given threshold and that has been verified. Processing continues to 212.

At 212, the results (e.g., the match results or the filtered match results) are output. The output can include an electronic message transmitted to a mobile device, an email sent automatically from the system to an email account, a synthesized voice call, audio output from a device, a printed output, or an electronic message transmitted to another system, or a display of the output information on a display device.

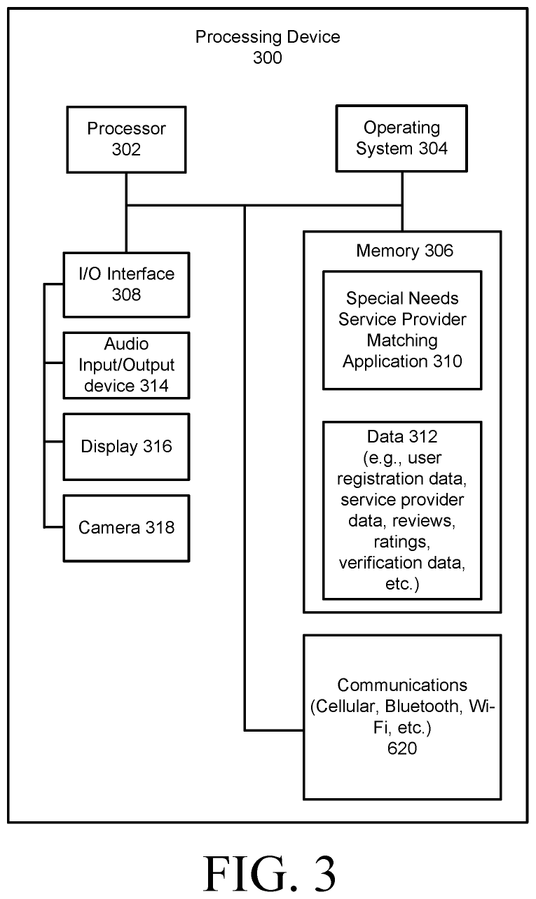

FIG. 3 is a block diagram of an example processing device 300 which may be used to implement one or more features described herein. In one example, device 300 may be used to implement a computer device, e.g., a server device (e.g., 114 of FIG. 1), and perform appropriate method implementations described herein (e.g., one or more of 202-210). Device 300 can be any suitable computer system, server, or other electronic or hardware device. For example, the device 300 can be a mainframe computer, desktop computer, workstation, portable computer, laptop computer, or electronic device (portable device, mobile device, cell phone, smart phone, tablet computer, television, TV set top box, personal digital assistant (PDA), media player, game device, wearable device, etc.). In some implementations, device 300 includes a processor 302, an operating system 304, a memory 306, and input/output (I/O) interface 308.

Processor 302 can be one or more processors and/or processing circuits to execute program code and control basic operations of the device 300. A “processor” includes any suitable hardware and/or software system, mechanism or component that processes data, signals or other information. A processor may include a system with a general-purpose central processing unit (CPU), multiple processing units, dedicated circuitry for achieving functionality, or other systems. Processing need not be limited to a particular geographic location or have temporal limitations. For example, a processor may perform its functions in “real-time,” “offline,” in a “batch mode,” etc. Portions of processing may be performed at different times and at different locations, by different (or the same) processing systems. A computer may be any processor in communication with a memory.

Memory 306 is typically provided in device 300 for access by the processor 302 and may be any suitable processor-readable storage medium, e.g., random access memory (RAM), read-only memory (ROM), Electrical Erasable Read-only Memory (EEPROM), Flash memory, etc., suitable for storing instructions for execution by the processor, and located separate from processor 302 and/or integrated therewith. Memory 306 can store software operating on the server device 300 by the processor 302, including an operating system 304, one or more applications 310, and data 312. In some implementations, applications 310 can include instructions that enable processor 302 to perform the functions described herein, e.g., some or all of the methods of FIGS. 2 and/or 6.

For example, applications 310 can include a special needs service provider matching application. Other applications or engines 314 can also or alternatively be included in applications 310, e.g., email applications, SMS and other phone communication applications, web browser applications, media display applications, communication applications, web hosting engine or application, social networking engine or application, etc. Any of software in memory 304 can alternatively be stored on any other suitable storage location or computer-readable medium. In addition, memory 304 (and/or other connected storage device(s)) can store images, video, and other instructions and data used in the features described herein. Memory 304 and any other type of storage (magnetic disk, optical disk, magnetic tape, or other tangible media) can be considered “storage” or “storage devices.”

I/O interface 308 can provide functions to enable interfacing the server device 300 with other systems and devices. For example, network communication devices, storage devices (e.g., memory and/or database), and input/output devices can communicate via interface 308. In some implementations, the I/O interface 308 can connect to interface devices including input devices (keyboard, pointing device, touchscreen, microphone, camera, scanner, etc.) and/or output devices (display device, speaker devices, printer, motor, etc.). Audio input/output device 314 (e.g., microphone and speaker), display device 316 and camera device 318 are examples of input/output devices that may be used to capture input (microphone and/or camera) and to provide output (display and speaker). Display device 316 can be connected to device 300 via local connections (e.g., display bus) and/or via networked connections and can be any suitable display device, some examples of which are described below.

For ease of illustration, FIG. 3 shows one block for each of processor 302, memory 306, I/O interface 308, and software block 310. These blocks may represent one or more processors or processing circuitries, operating systems, memories, I/O interfaces, applications, and/or software modules. In other implementations, device 300 may not have all of the components shown and/or may have other elements including other types of elements instead of, or in addition to, those shown herein. While 114 is described as performing operations as described in some implementations herein, any suitable component or combination of components of system 114 or similar system, or any suitable processor or processors associated with such a system, may perform the operations described.

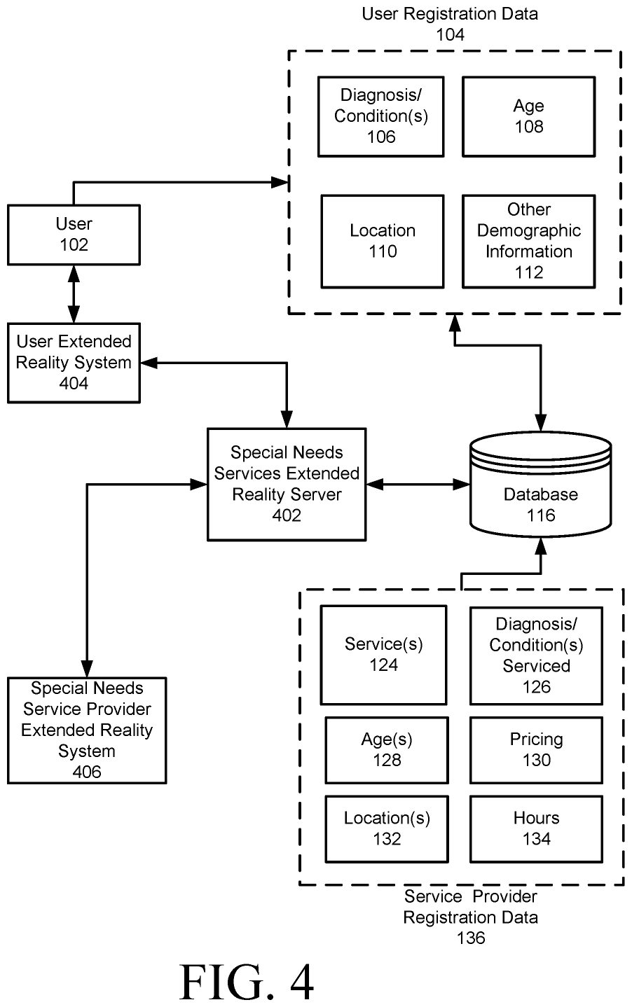

FIG. 4 is a block diagram of an example system for remotely providing and monitoring services for individuals with special needs via extended reality systems in accordance with some implementations. In particular, in addition to the components mentioned above in connection with FIG. 1, FIG. 4 shows a special needs services extended reality server 402, a user system 404, and a special needs service provider system 406.

The special needs services extended reality server 402, the user system 404, and the special needs service provider system 406 operate in conjunction to permit a service provider using the special needs service provider system 406 to provide and monitor services via extended reality to a person with special needs using the user system 404 via the special needs services extended reality server 402. These three components provide an end-to-end system for the remote provision and monitoring of special needs services such as education, socialization, physical therapy, occupational therapy, psychological therapy, or other therapies, treatments, or experiences.

Service providers for persons with special needs such as educators and therapists can utilize the environment of FIG. 4 for provision and/or monitoring of extended reality services for people with special needs as discussed above. Extended reality (or XR) refers collectively to a group of technologies such as virtual reality (VR), augmented reality (AR), and mixed reality (MR). In combination with other technological advances such as increased network data throughput, extended reality will rapidly become available commercially to users in educational, clinical, and home settings. Extended reality can be provided as a cloud service similar to the way some entertainment media such as music and movies are provided.

In addition to extended reality, artificial intelligence (AI) can provide an adaptive and more personalized educational or therapeutic experience for users. For example, an AI avatar or persona could interact with a user in an extended reality immersive experience and provide interaction that could be used for therapy or education exercise or practice sessions, while also providing a safe environment that protects the privacy of the user (e.g., student or patient) with special needs.

Extended reality can include real-and-virtual combined environments and human-machine interactions generated and facilitated by computer technology and wearables. Extended reality can range from partial sensory inputs to more full sensory immersive virtuality.

Extended reality includes a range of reality/virtual experiences from “completely real” to “completely virtual.” Extended reality systems can provide for the extension of human experiences especially relating to the senses of existence (as represented by VR) and the acquisition of cognition (as represented by AR). Extended reality systems include immersive technologies that extend the reality a user experiences by either blending the virtual and “real” worlds or by creating a fully immersive virtual experience.

Augmented Reality (AR)

In an augmented reality system, virtual information and objects can be overlaid on a representation of the real world. An AR experience can enhance the representation of the real world through digital details such as images, text, sound, haptic feedback, and/or animation. AR systems can include glasses, screens, tablets, and smartphones. Accordingly, users are not isolated from the representation of the real world and can still interact and essentially see what's going on in the real-world representation (e.g., what's going on in front of them).

Virtual Reality (VR)

In contrast to augmented reality systems, a virtual reality system provides users with an experience in which the users are fully immersed in a simulated digital environment. For example, users typically use a VR headset or head-mounted display to get a 360-degree view of an artificial world that essentially tricks the brain into believing that the user is performing an action or experiencing something, e.g., walking on a beach, flying through a city, or the like.

Mixed Reality (MR)

In a mixed reality system, digital and real-world objects can co-exist and interact with one another in real-time. Mixed reality is sometimes referred to as hybrid reality. Mixed reality utilizes an MR headset and typically more processing power than VR or AR. An example of mixed reality is Microsoft's HoloLens, which permits a user to place digital objects into the room the user is standing in and gives the user the ability to spin it around or interact with the digital object in any way possible.

Some implementations can include using extended reality to provide and/or monitor educational or therapeutic services for one or more persons with special needs. An advantage of using extended reality systems for special needs services is that the extended reality systems can stimulate multiple senses in a carefully controlled fully or partially artificial environment. For example, a user (e.g., a person with special needs) could wear a full-body suit (e.g., a Teslasuit manufactured VR Electronics Ltd. Of London, England) to provide haptic feedback, enhancing the immersive experience for a user through the sense of touch. The full body suits can also be equipped with an array of biometric sensors enabling the user's heartbeat, perspiration, and other stress indicators to be measured. The monitoring of these stress indicators (or other physiological parameters of the user) can help a service provider (e.g., and educator, therapist, or other professional) monitor the user (e.g., a person with special needs) and adapt or adjust the education or therapy protocol for maximum effectiveness for the user.

Extended reality can help lower the cost of treatment for a user by making the education or therapy environments virtual and therefore reducing the cost to travel to a fixed, physical location and also reducing the cost of maintaining a fixed physical location for the service provider. Additionally, because the extended reality system can permit the user to be placed virtually into a simulated location, the service provider can work with the user in numerous simulated environments (e.g., a school, a grocery store, a doctor's office, etc.) without having to spend the time and money to physically be present in the various locations.

For training or providing therapy for people with special needs, an extended reality system can be used to safely simulate any number of hazardous or stressful situations or conditions and monitor the way the user responds to them. Also, by being a computer-controlled system, an extended reality simulation can be very quickly terminated if it starts to cause excessive anxiety or other undesired reaction in the user, whereas it may be more difficult and stressful to try to exit a physical situation that is causing stress.

In some implementations, an extended reality special needs services system can effectively take total control of a user's senses (sight and hearing, particularly) to create a totally immersive experience that places the user in a fully virtual environment that would feel realistic to the user. Some extended reality systems can include simulating touch, taste and smell, and sensations such as hot or cold to create digital environments that appear completely real to all five senses simultaneously.

Full sensory simulation can provide a dematerialized educational/therapy environment where the physical structure of the educational or treatment facility effectively vanishes from user's lives as entirely interactive and collaborative working environments are possible for users in any area of the world by simply putting on a headset (or other device) and using other hardware for the type of extended reality educational or therapeutic task at hand.

Extended reality special needs services can include simulated social interactions that may be useful to permit a person with special needs to learn and develop social skills within a simulated environment that can be controlled to provide the most effective experience for the user. For example, extended reality systems can permit users to virtually meet, talk or play in virtual environments. Extended reality educational and/or therapeutic systems can permit users to build and share collaborative online worlds where they can hang out, play games, or work together on collaborative projects. This kind of social therapy or education can be used to help people with certain special needs become more social and interact with others in an environment that can be controlled and is safe for the users.

Some implementations can include artificial intelligence (or AI) such as a machine learning model as described herein. The use of a machine learning model in special education can permit automatic analysis of student data (e.g., video, audio, motion capture information, etc.) to identify problem areas. In some implementations, the machine learning model can be trained to detect disability or level of disability. For example, when an extended reality system is being used for teaching or therapy, a machine learning model trained to recognize disabilities can monitor data about the person with special needs from one or more sensors (e.g., video, audio, motion, location, etc.). For example, a machine learning model can be trained to analyze audio data, video of patient movement (e.g., extremity movements, expressions, nonverbal ques, etc.), or other data to predict a diagnosis. An example of a similar type of analysis performed in a lab setting was described in Kojovic, N., Natraj, S., Mohanty, S. P. et al. Using 2D video-based pose estimation for automated prediction of autism spectrum disorders in young children. Sci Rep 11, 15069 (2021), which is incorporated herein by reference. While the paper is different from the disclosed subject matter in the present application in a number of ways, such as a limited research environment and not being incorporated as a background monitoring and diagnosis prediction system as part of a broader virtual therapy, evaluation, or educational system, the paper does demonstrate that this type of machine learning approach to diagnostics is valid.

Also, a machine learning model can be trained to aid learning as described herein. In another example, a machine learning model can be used as part of a system to predict a diagnosis or to confirm a diagnosis, IQ, cognitive potential, potential, or the like. The machine learning model as described herein can be used in a manner analogous to the manner in which an optometrist uses a device and software to identify a prescription or other issues then they confirm it with optometrist led eye exam. Likewise, a computer software system including ML or AI, etc. can run a learner through a series of interactions then identify IQ, diagnosis, issues, etc. and then a live person can confirm by usual methods (reach out for clarification). A ML model can be used for progress monitoring in addition to diagnosis. For example, a ML model can be trained to detect cognitive ability exclusive of a current teacher's teaching system, style, prejudices, limitations, etc. Some learners learn differently but aren't cognitively impaired. Once the system identifies that the learner can actually do the work, it may be able to provide the teacher with suggestions on how this child could learn. Another example use for an ML model is to train the model to compare input from a person being evaluated or monitored against the “norm” to predict a diagnosis.

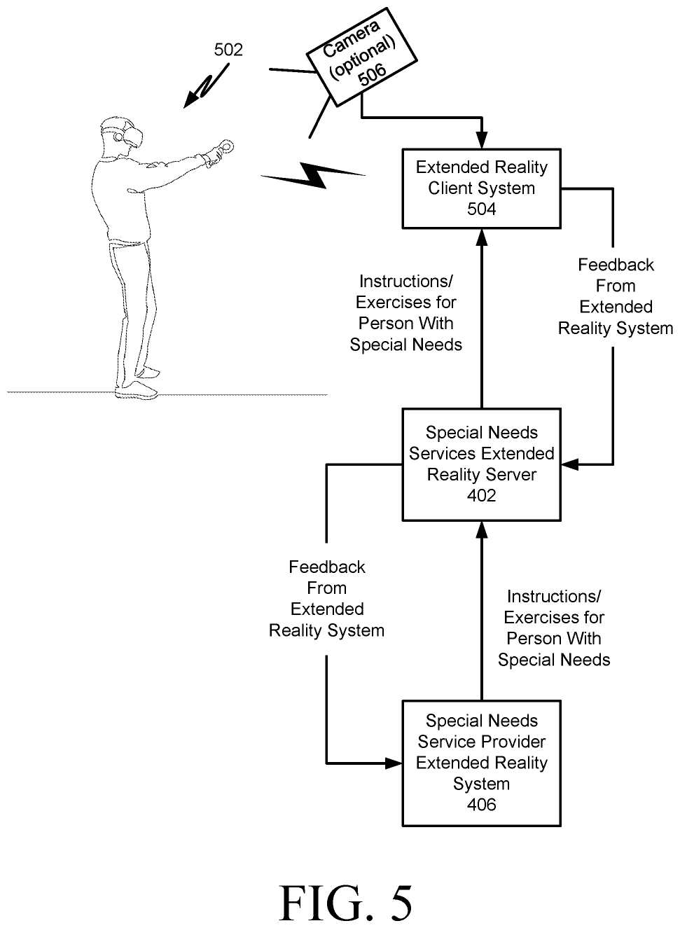

FIG. 5 is a block diagram of an example system for remotely providing and monitoring extended reality services for individuals with special needs in accordance with some implementations. In particular, FIG. 5 shows extended reality hardware 502 of a user, an extended reality user system 504, and an optional camera 506 (or other sensor).

In operation, a user (e.g., a person with special needs receiving therapy or education services, etc.) uses the extended reality hardware 502 to experience an extended reality simulation scenario. It will be appreciated that additional extended reality hardware (e.g., similar to 502) could be used by the special needs service provider, a parent, or other person to observe or interact with the user in the extended reality simulation scenario.

The extended reality hardware 502 can include one or more sensory displays, audio output devices, audio input devices, tracking devices, and input devices. For example, one of the basic goals of an extended reality system can be to provide the user's senses with information from the computer-generated reality in much the same way as a person experiences the real world. Because most people have two eyes, a natural way to see the world requires not one computer display, but two. In extended reality systems, a common way to produce a realistic 3D view of a virtual world is to place a small display in front of each eye or provide lens to focus each eye on a portion of a single display. Each eye of the user experiences the perspective that the corresponding eye would see in an actual environment. Such a system is sometimes called a binocular head-mounted display (HMD).

Similar to the having two eyes, most people also have two ears. Thus, stereophonic sound can be provided in an extended reality system. As two visual perspectives make a 3D view, two audio perspectives can make a 3D soundscape. By using headphones and presenting the correct acoustical perspectives to each ear, many of the spatial aspects of sounds can be preserved. HMDs often have headphones built into them. An extended reality system can also include one or more microphones to detect sound coming from the user. Additional sensory stimulation devices can be used to stimulate other senses such as haptic, olfactory, temperature, etc.

The extended reality user system 504 can include specialized hardware for the demands of extended reality. For example, the computer system may need fast processors and one or more graphics accelerator cards. The extended reality user system 504 can also include a 3D sound card to provide the user with a sense of position for a number of independent sound sources.

The extended reality user system 504 can also include a tracking system to measure position and orientation of one or more parts of a user's body (e.g., head, hands, arms, torso, legs, etc.). For example, from the position and orientation of the user's head, the extended reality user system 504 can determine how to display the virtual world so that it seems the user is in it as opposed to watching it on television. When the user turns his/her head the head tracker senses the change in position and adjusts the displays accordingly.

The head tracker needs to be capable of taking a measurement of position and orientation at a rate sufficient to accurately track the movements of the user's body (e.g., at least 20 times every second).

The extended reality user system 504 can also include one or more input devices. The input devices can be used to communicate user intentions and actions within the virtual world of extended reality. Since it is often difficult to touch-type or use a mouse while standing up and wearing a head-mounted display, other types of input systems are used instead of or in addition to keyboards and mice. For example, the extended reality user system 504 can also include one or more wands. A wand is basically a hand-held joystick with a number of buttons on it. Wands often include a position and orientation tracker which permits a user to pick up and rotate objects in the virtual world. A wand can be used in extended reality in much the same way a mouse is used on a computer desktop. Moving the wand in space can move a 3D pointer in the virtual environment. A user can also click and drag virtual objects, but instead of just moving them vertically and horizontally, the user can also move them in depth and rotate them about all three axes. Wands which can move objects in the X, Y, and Z directions and also rotate them about the X, Y, and Z axes are sometimes called six-dimensional or 6D controllers.

Because distances can be arbitrarily large in a virtual environment (and trackers have a limited range) it is not usually practical to travel through an extended reality simulation on foot. Accordingly, one or more wand buttons are often used for “flying”: a user pointing his/her wand or head in the direction of desired travel, and then pressing the “fly” button.

The extended reality user system 504 can also include one or more glove input devices. Glove input devices are similar to wands, only more complex. Glove input devices typically include a tracker to sense the position and orientation of the user's hand, and flex sensors to measure the bend of the user's fingers.

The extended reality user system 504 can also include an optional camera 506 can be used to provide an outside view of the user while engage in an extended reality simulation. This exterior view can be used by a system (e.g., 402 or 504) or a service provider to help evaluate or observe the user during an extended reality task or experience.

In operation, a service provider can provide instructions or an exercise (or task) to the person with special needs from the special needs service provider system 406 to the special needs services extended reality server 402 and on to the extended reality client system 504. Feedback from the extended reality client system 504 can be provided to the special needs services extended reality server 402 and on to the special needs service provider system 406.

The feedback can include a third-party view of the user in the extended reality simulation, the movement information of the user within the extended reality simulation, sound from the user, video of the user capture by camera 506 or other feedback indicating a parameter of the user's activity in attempting the instructions or exercise, etc.

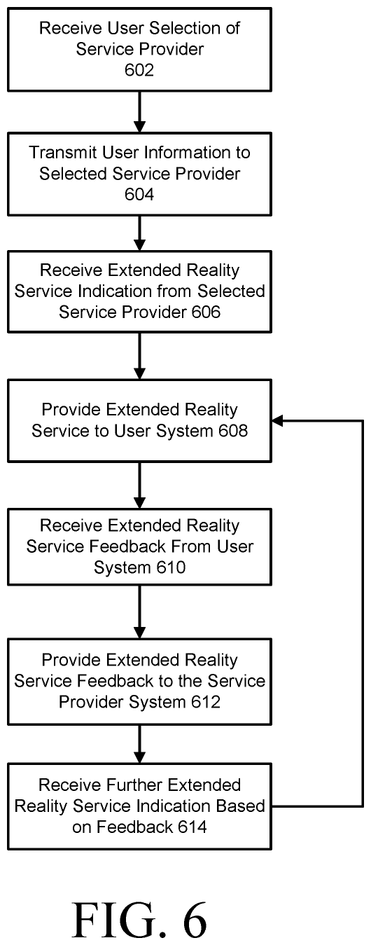

FIG. 6 is a flowchart of an example method 600 for providing extended reality services for individuals with special needs in accordance with some implementations. The method begins at 602, where a selection of a service provider is received. The service provider selection could be based, for example, on a recommendation from process 200 described above. The selection of a service provider could be made by a person with special needs or by another person on behalf of the person with special needs (e.g., by a parent, relative, or other service provider). Receiving the selection of the service provider includes receiving, at the special needs server, an electronic indication of an identifier of a service provider within the special needs services extended reality system. Processing continues to 604.

At 604, user information is transmitted to the selected service provider. For example, user information such as diagnoses/conditions, age, location, or other data is transmitted to the selected service provider system from the special needs services server. Processing continues to 606.

At 606, an extended reality service indication is received. For example, an indication of an extended reality service (e.g., an instruction, a task, an exercise, etc.) is received at the special needs services server from the selected service provider system. The indication can include an instruction, lesson, or practice exercise for the user to perform within the extended reality environment. Processing continues to 608.

At 608, the extend reality service indication is provided to the user system. For example, the special needs services server can provide the extended reality service indication to the extended reality user system for presentation to the user. The indication can be in the form of the extended reality simulation as generated by the special needs services server or can be a request for the user system to generate and present the extended reality service indication. Processing continues to 610.

At 610, feedback from the extended reality user system is received at the special needs services server. For example, the feedback can include a simulated third-party view of the user in the extended reality simulation, movement information of the user within the extended reality simulation, sound from the user, video of the user capture by camera 506 or other feedback indicating a parameter of the user's activity in attempting the instructions or exercise, etc. Some implementations can include sensors or technology similar to that used for measuring sports swings etc. Processing continues to 612.

At 612, the feedback from the extended reality user system is provided to the service provider system. For example, the feedback from step 610 is provided to the service provider system. The service provider system can include an extended reality system and the feedback can be presented in real time (or near real time) such that the service provider can observe or interact with the user within the extended reality simulation. In another mode, the feedback can be presented at a time different than the user was participating in the extended reality exercise or lesson. Processing continues to 614.

At 614, a further extended reality service indication based on the feedback is received from the service provider system. For example, if the feedback shows that the user successfully completed an exercise or task in the extended reality system, the further extended reality service indication could include a subsequent exercise or lesson in a course of education or therapy determined by the service provider or the service provider system. In another example, if the user failed to complete the exercise or lesson or was struggling with the exercise or lesson, the further extended reality service indication could include a remedial exercise or lesson or one in which the user could build skills necessary for successfully completing the original exercise or lesson. The further extended reality service indication could include an indication to repeat a given exercise or lesson to help the user build skills and practice the exercise or lesson. In another example, the feedback can be analyzed by a model trained to recognize disabilities. The model can then provide predictions about whether one or more given disabilities appear to be present in a person for which the feedback is associated with. The predictions about detected disabilities can be used to generate warnings and/or recommendations for service providers and/or parents or guardians of the person with special needs. Processing then returns to 608. A loop of extended reality indications, feedback, and further extended reality indications is created.

### Example Usage Scenarios

In one example scenario, the extended reality special needs services system discussed above could be used to demonstrate tasks such as teeth brushing, etc. Extended reality could also be used to show how to do different tasks (e.g., speech therapy tasks, occupational therapy tasks, physical therapy tasks, etc.) and to monitor performance of the tasks to measure growth and progress of the user.

Some implementations can include a system that can take in user information such as diagnostic information and give a service provider recommendation, and if selected, connect the user with the selected service provider and provide lessons or therapy via the extended reality system and monitor progress through the extended reality system.

Some implementations can include gamification of therapy tasks within the extended reality system so that the user is motivated and encouraged to make progress in the educational or therapeutic tasks to achieve success within a game.

In another example usage scenario, some kids can't be integrated with others because of significant issues, however the extended reality system could be used to help reduce sensory sensitivities by introducing triggers in a measured or controlled way. Also, the extended reality system could be used to filter certain sensory stimulations to reduce triggering sensory issues and introduce the triggers in a controlled manner. Some implementations could even be used for food triggers to help a user overcome a sensitivity to a given food or foods.

In another usage example, an extended reality system can be used for virtual integration through extended reality simulated exposure (e.g., visiting doctor or dentist virtually in a controlled way time limited, and slow introduction to place).

While this application discusses uses for people with special needs, it will be appreciate that the extended reality education/therapy system could be used in regular education (e.g., language learning, music education, sports, etc.).

Some implementations can include privacy protection measures to protect the privacy of the users and the service providers as extended reality systems may collect and process a large amount of very detailed and personal data about a user (e.g., actions, movements, even expressions and emotions). For example, a user may be able to select for the system to not store any personally identifiable information, etc.

One or more methods described herein (e.g., methods 200 or 600) can be implemented by computer program instructions or code, which can be executed on a computer. For example, the code can be implemented by one or more digital processors (e.g., microprocessors or other processing circuitry), and can be stored on a computer program product including a non-transitory computer readable medium (e.g., storage medium), e.g., a magnetic, optical, electromagnetic, or semiconductor storage medium, including semiconductor or solid state memory, magnetic tape, a removable computer diskette, a random access memory (RAM), a read-only memory (ROM), flash memory, a rigid magnetic disk, an optical disk, a solid-state memory drive, etc. The program instructions can also be contained in, and provided as, an electronic signal, for example in the form of software as a service (SaaS) delivered from a server (e.g., a distributed system and/or a cloud computing system). Alternatively, one or more methods can be implemented in hardware (logic gates, etc.), or in a combination of hardware and software. Example hardware can be programmable processors (e.g., Field-Programmable Gate Array (FPGA), Complex Programmable Logic Device), general purpose processors, graphics processors, Application Specific Integrated Circuits (ASICs), and the like. One or more methods can be performed as part of or component of an application running on the system, or as an application or software running in conjunction with other applications and operating system.

One or more methods described herein can be run in a standalone program that can be run on any type of computing device, a program run on a web browser, a mobile application (“app”) run on a mobile computing device (e.g., cell phone, smart phone, tablet computer, wearable device (wristwatch, armband, jewelry, headwear, goggles, glasses, etc.), laptop computer, etc.). In one example, a client/server architecture can be used, e.g., a mobile computing device (as a client device) sends user input data to a server device and receives from the server the final output data for output (e.g., for display). In another example, all computations can be performed within the mobile app (and/or other apps) on the mobile computing device. In another example, computations can be split between the mobile computing device and one or more server devices.

The special needs service provider matching system 114 can include one or more machine learning models such as a neural network. The training of the model can include user-specific training of the neural network(s) based on past user service provider choices, etc. The training approach mentioned above could include associated weights for predictions. In some implementations, the trained model is trained offline and/or online, e.g., if the user selects a service provider that was predicted, that can act as positive reinforcement to the model, while if the user chooses a different service provider that can serve as negative reinforcement. Online training can permit the machine learning model to dynamically adjust predictions of service providers or service provider recommendations. Online training permits the system to adjust the selection process over time.

In some implementations, a single neural network can be trained based on data with weights assigned to training inputs.

The model form or structure may specify connectivity between various nodes and organization of nodes into layers. For example, nodes of a first layer (e.g., input layer) may receive data as input data or application data. Subsequent intermediate layers may receive as input output of nodes of a previous layer per the connectivity specified in the model form or structure. These layers may also be referred to as hidden layers. A final layer (e.g., output layer) produces an output of the machine-learning application. For example, the output may provide one or more special needs service provider recommendations or suggestions.

In some implementations, the trained model may include weighted individual nodes and/or connections. A respective weight may be applied to a connection between each pair of nodes that are connected per the model form, e.g., nodes in successive layers of the neural network. In some implementations, respective weights may be randomly assigned, or initialized to default values. The model may then be trained, e.g., using data, to produce a result, where the training can include adjusting one or more of nodes, node structure, connections, and/or weights.

A model can include a loss function representing the difference between a predicted value and an actual label. The model can be trained to minimize the loss function. Training can include supervised, unsupervised, or semi-supervised learning techniques. In supervised learning, the training data can include a plurality of inputs and a corresponding expected output for each input. Based on a comparison of the output of the model with the expected output (e.g., computing the loss function), values of the weights are automatically adjusted, e.g., in a manner that increases a probability that the model produces the expected output when provided similar input (i.e., reduces the loss function). In unsupervised learning, models learn relationships between elements in a data set and classify raw data without the benefit of labeled training data. Semi-supervised learning can include a combination of supervised and unsupervised techniques, for example, a small amount of labeled training data and a large amount of unlabeled training data can be provided to a model for learning. Once the model is trained, it can be used to predict special needs service providers based on real-world data.

In some implementations, neural networks (as well as other learning algorithms) tend to produce a weighted set of choices. Some implementations can include performing the training step until the weight for the correct answer is a threshold value larger than the next option. By continually improving the data set, and by discarding incorrect decisions, there may be little downside to shipping any particular network. Performance can be analyzed by tracking how well any particular network is performing over time.

Although the description has been described with respect to particular implementations thereof, these particular implementations are merely illustrative, and not restrictive. Concepts illustrated in the examples may be applied to other examples and implementations.

Note that the functional blocks, operations, features, methods, devices, and systems described in the present disclosure may be integrated or divided into different combinations of systems, devices, and functional blocks as would be known to those skilled in the art. Any suitable programming language and programming techniques may be used to implement the routines of particular implementations. Different programming techniques may be employed, e.g., procedural or object-oriented. The routines may execute on a single processing device or multiple processors. Although the steps, operations, or computations may be presented in a specific order, the order may be changed in different particular implementations. In some implementations, multiple steps or operations shown as sequential in this specification may be performed at the same time.

It will be appreciated that the modules, processes, systems, and sections described above can be implemented in hardware, hardware programmed by software, software instructions stored on a nontransitory computer readable medium or a combination of the above. A system as described above, for example, can include a processor configured to execute a sequence of programmed instructions stored on a nontransitory computer readable medium. For example, the processor can include, but not be limited to, a personal computer or workstation or other such computing system that includes a processor, microprocessor, microcontroller device, or is comprised of control logic including integrated circuits such as, for example, an Application Specific Integrated Circuit (ASIC). The instructions can be compiled from source code instructions provided in accordance with a programming language such as Java, C, C++, C#.net, assembly or the like. The instructions can also comprise code and data objects provided in accordance with, for example, the Visual Basic™ language, or another structured or object-oriented programming language. The sequence of programmed instructions, or programmable logic device configuration software, and data associated therewith can be stored in a nontransitory computer-readable medium such as a computer memory or storage device which may be any suitable memory apparatus, such as, but not limited to ROM, PROM, EEPROM, RAM, flash memory, disk drive and the like.

Furthermore, the modules, processes systems, and sections can be implemented as a single processor or as a distributed processor. Further, it should be appreciated that the steps mentioned above may be performed on a single or distributed processor (single and/or multi-core, or cloud computing system). Also, the processes, system components, modules, and sub-modules described in the various figures of and for embodiments above may be distributed across multiple computers or systems or may be co-located in a single processor or system. Example structural embodiment alternatives suitable for implementing the modules, sections, systems, means, or processes described herein are provided below.

The modules, processors or systems described above can be implemented as a programmed general purpose computer, an electronic device programmed with microcode, a hard-wired analog logic circuit, software stored on a computer-readable medium or signal, an optical computing device, a networked system of electronic and/or optical devices, a special purpose computing device, an integrated circuit device, a semiconductor chip, and/or a software module or object stored on a computer-readable medium or signal, for example.

Embodiments of the method and system (or their sub-components or modules) may be implemented on a general-purpose computer, a special-purpose computer, a programmed microprocessor or microcontroller and peripheral integrated circuit element, an ASIC or other integrated circuit, a digital signal processor, a hardwired electronic or logic circuit such as a discrete element circuit, a programmed logic circuit such as a PLD, PLA, FPGA, PAL, or the like. In general, any processor capable of implementing the functions or steps described herein can be used to implement embodiments of the method, system, or a computer program product (software program stored on a nontransitory computer readable medium).

Furthermore, embodiments of the disclosed method, system, and computer program product (or software instructions stored on a nontransitory computer readable medium) may be readily implemented, fully or partially, in software using, for example, object or object-oriented software development environments that provide portable source code that can be used on a variety of computer platforms. Alternatively, embodiments of the disclosed method, system, and computer program product can be implemented partially or fully in hardware using, for example, standard logic circuits or a VLSI design. Other hardware or software can be used to implement embodiments depending on the speed and/or efficiency requirements of the systems, the particular function, and/or particular software or hardware system, microprocessor, or microcomputer being utilized. Embodiments of the method, system, and computer program product can be implemented in hardware and/or software using any known or later developed systems or structures, devices and/or software by those of ordinary skill in the applicable art from the function description provided herein and with a general basic knowledge of the software engineering, image processing and/or machine vision arts.

Moreover, embodiments of the disclosed method, system, and computer readable media (or computer program product) can be implemented in software executed on a programmed general-purpose computer, a special purpose computer, a microprocessor, a network server or switch, or the like.

It is, therefore, apparent that there is provided, in accordance with the various embodiments disclosed herein, methods, systems and computer readable media for special needs service provider matching.

While the disclosed subject matter has been described in conjunction with a number of embodiments, it is evident that many alternatives, modifications and variations would be, or are, apparent to those of ordinary skill in the applicable arts. Accordingly, Applicants intend to embrace all such alternatives, modifications, equivalents and variations that are within the spirit and scope of the disclosed subject matter.

What is claimed is:

1. A method comprising:
obtaining user registration data;
obtaining service provider registration data;
receiving a query;
matching a combination of query data and user registration data with the service provider data; and
outputting a result of the matching, wherein the result includes one or more records that match within a given threshold.

2. The method of claim 1, further comprising filtering the result of the matching based on one or more third-party data items and outputting the filtered results.

3. The method of claim 2, wherein the user registration data includes one or more of a diagnosis or a condition corresponding to a special need of a person.

4. The method of claim 3, wherein the combination includes the diagnosis or the condition.

5. The method of claim 4, wherein the diagnosis or the condition is independent of a service provided by the service provider, and wherein the matching results include service provider data indicating that the matching service providers provide the service from the query to people having the diagnosis or the condition.

6. The method of claim 5, wherein the third-party data includes one of a rating above a rating threshold or a verification, wherein the rating threshold is included as part of the query, and the verification indicates that the service provider was verified by a third-party.
